(19) United States
(12) Patent Application Publication
(10) Pub. No.: US 2016/0300013 A1
(43) Pub. Date: Oct. 13, 2016

(54) METHOD FOR SIMULTANEOUS DETECTION OF GENOME-WIDE COPY NUMBER CHANGES, cnLOH, INDELS, AND GENE MUTATIONS

(71) Applicant: Agilent Technologies, Inc., Loveland, CA (US)

(72) Inventors: Ashutosh, San Jose, CA (US); Devendra Joshi, Cupertino, CA (US); Arjun Vadapalli, Sunnyvale, CA (US); Jayati Ghosh, Sunnyvale, CA (US)

(21) Appl. No.: 14/684,242

(22) Filed: Apr. 10, 2015

(51) Int. Cl. G06F 19/22; C12Q 1/68

(52) U.S. Cl. CPC G06F 19/22; C12Q 1/6869

(57) ABSTRACT

Provided herein are methods for simultaneously identifying genomic copy number variations (CNVs) and sequence variations in an enriched genomic sample and compositions, systems, and kits for performing such methods. In some aspects, the methods include: (a) obtaining a plurality of sequence reads from an enriched genomic sample that includes a plurality of genomic backbone regions and a plurality of genomic mutation regions of interest in a genomic locus of a subject; (b) obtaining a plurality of sequence reads from corresponding genomic backbone regions and genomic mutation regions of at least one reference genomic sample; (c) assembling the plurality sequence reads from the enriched genomic sample and the at least one reference genomic sample; and (d) determining, based on computational analysis of the assembly, whether the genomic locus has a copy number variation (CNV) and/or a sequence variation. The present disclosure further includes aspects in which the methods are performed by a computer and provide an output to a user identifying a genomic CNV and/or sequence variation.

### BACKGROUND

The human genome has several types of variation which confer genetic differences between individuals. Single nucleotide polymorphisms are sites of single base changes that vary from a reference genome. Copy number variants (CNVs) are larger regions of DNA which are duplicated or deleted with respect to a reference genome. Additionally, somatic alterations of the genome occur within subpopulations of an individual's cells or tissues, e.g., base mutations or small insertions-deletions (indels).

The technology platforms available to identify CNVs include fluorescence in situ hybridization (FISH), array comparative genomic hybridization (aCGH) and more recently next generation sequencing. More mature platforms such as FISH and aCGH suffer from low resolution of genomic regions. The rapid development of low cost short-read sequencing technologies has paved the way to detect mutations and high resolution structural variation detection in a single experiment. Using next generation sequencing technologies the user can either sequence the entire genome or sequence regions captured with target enrichment assays (such as SureSelect™). The choice between whole genome sequencing (WGS) and target enrichment-based sequencing depends on balancing cost and sequencing output. The current cost of whole genome sequencing to can be over a thousand dollars and increase the computational time to analyze the data.

A more economical alternative to WGS is sequencing of small gene panels or an entire exome that represents a highly enriched subset of the human genome. To implement such alternatives, improvements in the simultaneous detection of multiple genetic variations and/or alterations in the genome of an individual (e.g., SNPs, CNVs, indels, etc.), for example in the diagnosis and/or analysis of the progression of a disease.

### SUMMARY

Provided herein are methods, systems, and kits for the simultaneous detection of multiple genomic differences or alterations in one or more cells from an subject, including genomic copy number variations (CNVs), copy-neutral loss-of-heterozygosity (cnLOH), indels, mutations, and translocations.

Aspects of the present disclosure are drawn to methods for detecting genomic alterations, comprising: (a) obtaining a plurality of sequence reads from an enriched genomic sample that includes a plurality of genomic backbone regions and a plurality of genomic mutation regions of interest in a genomic locus of a subject; (b) obtaining a plurality of sequence reads from corresponding genomic backbone regions and genomic mutation regions of at least one reference genomic sample; (c) assembling the plurality of sequence reads from the enriched genomic sample and the at least one reference genomic sample; and (d) determining, based on computational analysis of the assembly, whether the genomic locus has a copy number variation (CNV) or a sequence variation, or a CNV and a sequence variation. In certain embodiments, determining whether the genomic locus has a sequence variation comprises: (i) identifying genetic differences in the plurality of sequence reads of the enriched genomic sample as compared to the sequence reads from the enriched genomic sample; and (ii) determining which of the potential variants are true and which of the potential variants are artifacts by examining the sequence reads that make up each of the discrete sequence assemblies. In certain embodiments, determining whether the genomic locus has a sequence variation comprises using a SNPPET algorithm. In certain embodiments, determining whether the genomic locus comprises a sequence variation further comprises determining whether the genomic locus has a loss of heterozygosity (LOH), comprising: (i) identifying genetic differences in the sequence reads of the enriched genomic sample as compared to the sequence reads from the at least one reference genomic sample; and (ii) comparing the identified genetic differences to known frequencies of genetic differences in a population to identify a genomic region in the first genomic locus that has an LOH. In certain embodiments, determining whether the genomic locus has a CNV comprises: (i) comparing the log ratios of the number of sequence reads from the enriched genomic sample to the number of sequence reads of the at least one reference genomic sample across the first genomic locus; and (ii) determining a genomic location of one or more break points in the log ratios and selecting one or more of the break points that are statistically significant to detect regions in the first genomic locus that have a CNV. In certain embodiments, determining whether the genomic locus has a sequence variation comprises: (i) processing sequence reads from two different regions of the enriched genome which differ in at least one statistical property or characteristic to make them amenable to analysis. In certain embodiments, the genomic mutation regions of interest comprise high minor allele frequency single polynucleotide polymorphic sites. In certain embodiments, the method further comprises (e) outputting a report indicating whether the enriched genomic sample comprises a CNV or a sequence variation, or a CNV and a sequence variation. In certain embodiments, the report indicates whether the enriched genomic sample contains a mutation and provides publicly available information about the reference sequence. In certain embodiments, one or more of the genomic mutation regions are associated with cancer. In certain embodiments, the enriched genomic sample is from a human. In certain embodiments, the assembling uses graph theory. In certain embodiments, the assembling is done using a minimal de-Bruijn sequence. In certain embodiments, the assembling is done using a BEST theorem. In certain embodiments, the enriched genomic sample is obtained using baits designed to target locations in the genome based on known SNP allelic frequency and estimated properties of the genomic regions of interest in the reference genome.

Aspects of the present disclosure are drawn to kits for enriching a genomic sample comprising: a firs set of probes designed to hybridize to genomic backbone regions, and a second set of probes designed to hybridize to regions of interest for which genomic sequence information is desired, wherein the probes in the first and second set include at least one modification selected from the group consisting of: a capture agent, a barcode sequence, a restriction enzyme site, a primer binding sites, and any combination thereof.

Aspects of the present disclosure are drawn to systems for detecting genomic alterations comprising: a database configured to store reference sequence reads for one or more reference genomes; and a computing device configured to: (a) obtain a plurality of sequence reads from an enriched genomic sample that includes a plurality of genomic backbone regions and a plurality of genomic mutation regions of interest in a genomic locus of a subject; (b) obtain from the database a plurality of reference sequence reads from corresponding genomic backbone regions and genomic mutation regions of at least one reference genome; (c) assemble the plurality of sequence reads from the enriched genomic sample and the at least one reference genomic sample; and (d) determine, based on computational analysis of the assembly, whether the genomic locus has a copy number variation (CNV) or a sequence variation, or a CNV and a sequence variation.

Aspects of the present disclosure are drawn to computer readable media comprising: (a) a code sequence to obtain a plurality of sequence reads from an enriched genomic sample that includes a plurality of genomic backbone regions and a plurality of genomic mutation regions of interest in a genomic locus of a subject; (b) a code sequence to obtain a plurality of sequence reads from corresponding genomic backbone regions and genomic mutation regions of at least one reference genomic sample; (c) a code sequence to assemble the plurality of sequence reads from the enriched genomic sample and the at least one reference genomic sample; and (d) a code sequence to determine, based on computational analysis of the assembly, whether the genomic locus has a copy number variation (CNV) or a sequence variation, or a CNV and a sequence variation.

These and other features of the present teachings are set forth herein.

### BRIEF DESCRIPTION OF THE FIGURES

The skilled artisan will understand that the drawings, described below, are for illustration purposes only. The drawings are not intended to limit the scope of the present teachings in any way.

FIG. 1: Flow chart detailing aspects of the present disclosure according to one embodiment.

FIG. 2: Flow chart showing aspects of genomic enrichment probe (bait) design according to one embodiment.

FIG. 3. Bait design schema used for genomic sequence enrichment showing backbone, SNP, clinical gene (ClinGen) disease associated region, and targeted exon regions.

FIG. 4. Determination of copy number changes in Agilent SureCall software. Log₂ ratios of the sequencing read-depth of the sample versus the sequencing read-depth of the control are plotted along the chromosome. No copy number change corresponds to a log₂ ratio of 0, a one copy loss corresponds to a log₂ ratio of −1, a one copy gain corresponds to a log₂ ratio of 0.58.

FIG. 5. On-target coverage of the sequence reads from enriched genomic samples (see Examples section for description).

FIG. 6. Copy number data analysis in Agilent SureCall software (top panel) and aCGH copy number data analysis in Agilent CytoGenomics software (bottom panel) showing a 13 Mb deletion on chromosome 13 in Coriell sample NA08254. Each plus sign represents a raw data point. Note the higher data point density in specific regions of interest.

FIG. 7. Copy number data analysis in Agilent SureCall software (top panel) and aCGH copy number data analysis in Agilent CytoGenomics software (bottom panel) showing a 370 kb amplification on chromosome 6 in Coriell sample NA08254.

FIG. 8. Copy number and LOH data analysis in Agilent SureCall software data analysis showing UPD 15 in Coriell sample NA20409. The top panel shows the copy number data. Each cross represents a raw data point. The entire chromosome, except for a known common CNV close to the centromere, is diploid. The bottom panel shows the LOH data. Each dot represents the B allele frequency of a SNP. The distribution of frequencies show UPD of chromosome 15.

FIG. 9. NA20408 shows maternal uniparental disomy (UPD) in chromosome 15 which is associated with Prader-Willi syndrome.

FIG. 10. Identification of mutations and indels in Agilent SureCall software showing a 26-bp indel (on the right) and a heterozygous SNP (on the left) in the MECP2 gene of Coriell sample NA16382.

### DEFINITIONS

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present teachings, some exemplary methods and materials are now described.

The term “sample”, as used herein, relates to a material or mixture of materials, typically, although not necessarily, in liquid form, containing one or more analytes of interest.

The term “amplifying” as used herein refers to generating one or more copies of a target nucleic acid, using the target nucleic acid as a template.

As used herein, the term “single nucleotide polymorphism,” or “SNP” for short, refers to a single nucleotide position in a genomic sequence for which two or more alternative alleles are present at appreciable frequency in a population.

The terms “chromosomal alteration”, “chromosomal difference”, “chromosomal change” and the like refer to a difference between the chromosome of a test sample and the chromosome of a reference sample. Examples of chromosome alteration/differences/changes include copy number variations (CNVs)(e.g., duplications or deletions of all or part of a chromosome), chromosomal rearrangements (e.g., inversions, translocations), mutations, and small insertions-deletions (also referred to herein as “indels”), etc. Chromosomal alterations/differences/changes are understood by those having ordinary skill in the art, some of which are further defined below.

The term “loss of heterozygosity” or “LOH” for short, indicates that a region of a test genome has lost heterozygosity relative to a parent genome or to a representative reference genome. LOH may be caused by several biological mechanisms, e.g., deletion of one copy of a region of a diploid chromosome (or UniParental Disomy (UPD)), which can occur by trisomy within a fertilized egg followed by loss of one copy of the chromosome, known as “trisomy rescue”. In cancer tumors, LOH is frequently caused by a somatic chromosomal rearrangement.

The term “copy neutral loss of heterozygosity” or “cnLOH” refers to a region of a test genome that lacks heterozygosity but whose copy number is the same as a diploid reference genome. Copy neutral LOH can occur when both copies of a genomic region in a diploid genome are contributed by a single parent, by parental consanguinity, or by a gene conversion event in which a locus in a first chromosome of homologous chromosomes is replaced by the same locus in the second chromosome of the pair, leaving two copies of the second locus. Copy neutral LOH is common in both hematologic and solid tumors, and is thought to constitute 20 to 80% of the loss of heterozygosity observed in human tumors. Copy-neutral LOH cannot be detected by traditional CGH, FISH, or cytogenetics methods. A region that has lost heterozygosity can be identified as such because all the SNPs in the region are homozygous (i.e., from one parent or the other) rather than heterozygous. Copy neutral LOH is further described in: Mao et al., Curr Genomics, 2007, 8: 219-28; Gondek et al., Blood, 2008, 111: 1534-42; Beroukhim et al., PLoS Comput. Biol., 2006, 2:e41; Ishikawa et al., Biochem. Biophys. Res. Commun., 2005, 333:1309-14; and Lo et al., Genes Chrom. Cancer., 2008, 47: 221-37.

The term “data” refers to both raw data and processed data. Raw data may be processed, e.g., normalized, smoothed, filtered, etc., prior to use in the subject method using any suitable method (see, e.g., Quackenbush, Nat. Gen., 2002, Supp. 32; van Houte et al., BMC Genomics., 2009, 10:401; Staaf et al., BMC Genomics., 2007, 8:382, Staaf et al., BMC Bioinformatics., 2008, 9:409; Rigaill et al., Bioinformatics., 2008 24:768-74; Curry et al., Normalization of Array CGH Data, In Methods in Microarray Normalization, CRC Press, 2008, 233-244; hereby incorporated by reference herein for all data processing steps).

The term “obtaining” refers to any way of coming into possession of something, including accessing a data file in silico, as well as receiving a data file from a remote location.

The term “probability distribution function” is a continuous probability density function that identifies the probability of a value falling within a particular interval. A probability distribution clusters around a single mean value and describes the range of possible values that a random variable can attain and the probability that the value of the random variable is within any measurable subset of that range. Probability distribution functions include normal (i.e., Gaussian) distributions, although other distributions may be used. Methods for plotting a probability distribution function for data that forms a normal distribution are known. The probability that a hypothesis is true can be estimated using, e.g., Bayes' theorem, although other methods are known.

The term “confidence” refers to calculated estimate of the reliability of a determination. Confidence can be measured in any suitable way, e.g., using Bayes' theorem and expressed using, e.g., a p-value or a percentage or the like.

The term “enriching,” with respect to a genome, refers to the separation of one or more regions of a genome from the remainder of the genome to produce a product that is isolated from the remainder of the genome. Enriching may be done using a variety of methods including those described in, e.g., Hedges et al., Comparison of three targeted enrichment strategies on the SOLiD sequencing platform, PLoS One, 2011, 6: e18595; and Shearer et al., Solution-based targeted genomic enrichment for precious DNA samples, BMC Biotechnol., 2012, 12: 20. In some embodiments, enrichment is done using commercially available systems, e.g., SureSelect, SureSelectXT, or HaloPlex enrichment systems (Agilent Technologies, Inc.; Santa Clara, Calif.). Enriching can be performed using sequence specific capture agents, or polynucleotide “baits”, that hybridized to genomic targets in a fragmented genomic sample. The target/bait hybridization complexes can then be isolated from the genomic fragments that are not hybridized to a bait polynucleotide, thereby generating an enriched sample.

The term “enriched sample” refers to a sample that contains fragments of genomic DNA that have been isolated from the remainder of the genome. Enriched fragments can be of any length depending on the fragmentation method used. In certain embodiments, the fragments may be in the range of 100 bp to 1 kb in length, e.g., 200 bp to 500 bp in length, although fragments outside of this range may be used. Depending on how the fragmentation and/or enriching is done, for any one enriched region, the ends of the fragment molecules may be the same or different.

The term “genomic region,” as used herein, refers to a region of a genome, e.g., an animal or plant genome such as the genome of a human, monkey, rat, fish or insect or plant.

A “plurality” contains at least 2 members. In certain cases, a plurality may have at least 10, at least 100, at least 1000, at least 10,000, at least 100,000, at least 10⁶, at least 10⁷, at least 10⁸ or at least 10⁹ or more members.

The term “sequencing,” as used herein, refers to a method by which the identity of at least 10 consecutive nucleotides (e.g., the identity of at least 20, at least 50, at least 100 or at least 200 or more consecutive nucleotides) of a polynucleotide are obtained.

The term “next-generation sequencing” or “NGS” refers to the so-called parallelized sequencing-by-synthesis or sequencing-by-ligation platforms currently employed by Illumina, Life Technologies, and Roche, etc. Next-generation sequencing methods may also include nanopore sequencing methods or electronic-detection based methods such as Ion Torrent technology commercialized by Life Technologies.

The term “sequence reads” refers to the output of a sequencing run. Sequence reads are represented by a string of nucleotides. Sequence reads may be accompanied by metrics about the quality of the sequence. For example, each nucleotide in a sequence read may be associated with the confidence of the base call, i.e., a determination of whether a nucleotide is a G, A, T or C, for that nucleotide.

The term “sequence variant” refers to a nucleic acid sequence that is different from a reference sequence at minimum at one position. Examples of sequence variants include sequences containing SNPs and somatic mutations.

The terms “low frequency sequence variant,” “minority species” and “minority variant” refer to a variant sequence that is only present in a sample at a frequency of less than 10% (e.g., less than 5% or less than 1%), relative to the non-variant version of the sequence. In many cases, a low frequency sequence may be represented by a nucleotide substitution or indel in a gene, and the non-variant version of the sequence will be represented by a wild type allele of the same gene. Low frequency sequence variants can be generated by somatic mutations, for example.

The term “reference sequence” refers to a known sequence, e.g., a sequence from a public or in-house database, to which a candidate sequence can be compared.

As used herein, the term “assembling” refers to a multi-step process that involves: aligning sequences that represent fragments of a longer nucleic acid. In certain cases, assembling may involve merging the sequences in order to construct the sequence of a segment.

As used herein, the term “anchor” refers to a sequence that is present in longer sequences that can be used to align those sequences. In certain cases, an anchor may be sufficient to correctly align the longer sequences.

As used herein, the term “sequence contig” refers to a contiguous sequence of nucleotides that is produced by assembling overlapping sequences.

A used herein, the term “associated with cancer” refers to a genomic region, e.g., a gene, that contains mutations correlated with a cancerous phenotype. In some cases, the mutations are believed to have a causative role in cancer.

### DETAILED DESCRIPTION

Before the various embodiments are described, it is to be understood that the teachings of this disclosure are not limited to the particular embodiments described, and as such can, of course, vary. It is also to be understood that the terminology used herein is for the purpose of describing particular embodiments only, and is not intended to be limiting, since the scope of the present teachings will be limited only by the appended claims.

The section headings used herein are for organizational purposes only and are not to be construed as limiting the subject matter described in any way. While the present teachings are described in conjunction with various embodiments, it is not intended that the present teachings be limited to such embodiments. On the contrary, the present teachings encompass various alternatives, modifications, and equivalents, as will be appreciated by those of skill in the art.

Where a range of values is provided, it is understood that each intervening value, to the tenth of the unit of the lower limit unless the context clearly dictates otherwise, between the upper and lower limit of that range and any other stated or intervening value in that stated range is encompassed within the present disclosure.

The citation of any publication is for its disclosure prior to the filing date and should not be construed as an admission that the present claims are not entitled to antedate such publication by virtue of prior invention. Further, the dates of publication provided can be different from the actual publication dates which can need to be independently confirmed.

It must be noted that as used herein and in the appended claims, the singular forms “a,” “an,” and “the” include plural referents unless the context clearly dictates otherwise. It is further noted that the claims can be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which can be readily separated from or combined with the features of any of the other several embodiments without departing from the scope or spirit of the present teachings. Any recited method can be carried out in the order of events recited or in any other order which is logically possible.

One with skill in the art will appreciate that the present invention is not limited in its application to the details of construction, the arrangements of components, category selections, weightings, pre-determined signal limits, or the steps set forth in the description or drawings herein. The invention is capable of other embodiments and of being practiced or being carried out in many different ways.

Aspects of the present disclosure include methods for detecting genomic alterations in the genome of a subject. Compositions, system and kits for performing such methods are also provided.

Aspects of the present disclosure include methods for detecting genomic alterations in at least one genomic locus in the genome of a subject, including:

(a) obtaining a plurality of sequence reads from an enriched genomic sample (e.g., for at least a first genomic locus) that includes a plurality of genomic backbone regions and a plurality of genomic mutation regions of interest in a genomic locus of a subject;

(b) obtaining a plurality of sequence reads from corresponding genomic backbone regions and genomic mutation regions of at least one reference genomic sample;

(c) assembling the plurality sequence reads from the enriched genomic sample and the at least one reference genomic sample; and

(d) determining, based on computational analysis of the assembly, whether the genomic locus has a copy number variation (CNV), a loss of heterozygosity (LOH), a SNP, a base mutation, or an insertion-deletion (indel).

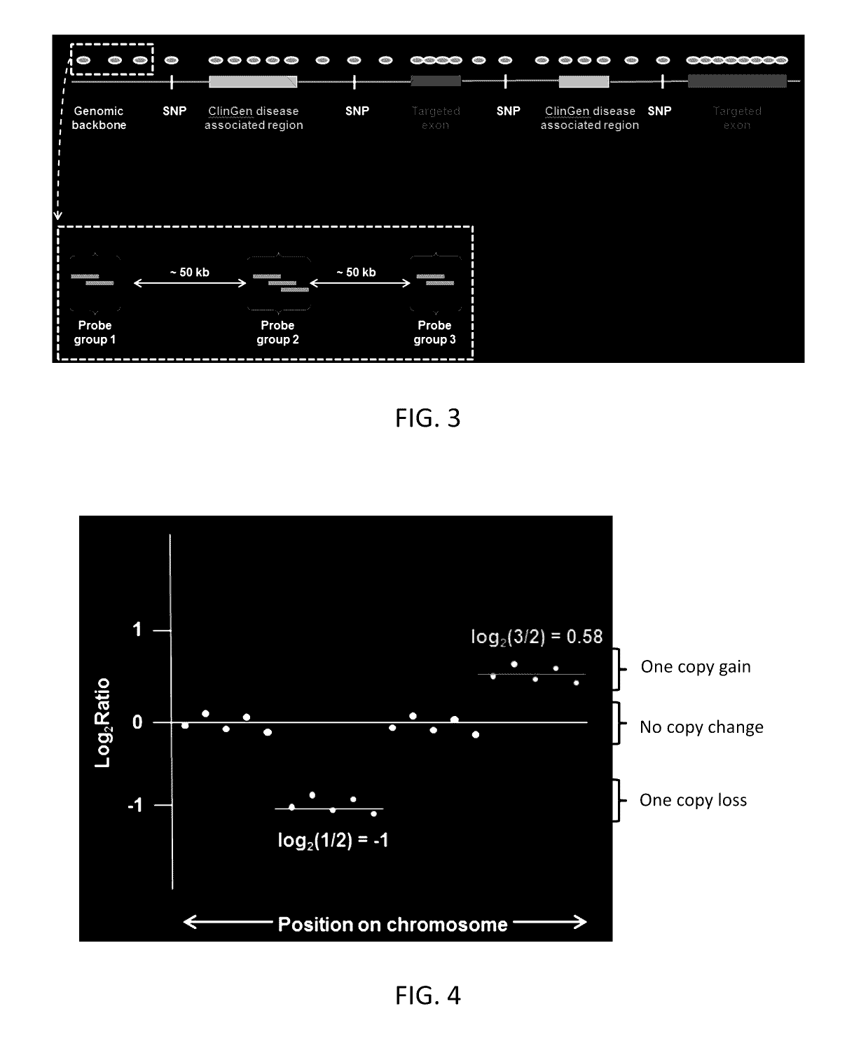

As noted above, enriched genomic samples (for both the test/sample and reference genomes) include a plurality of genomic backbone regions and a plurality of genomic mutation regions of interest in a genomic locus of a subject. Genomic backbone regions are generally used to determine the genome-wide copy number of the locus of interest whereas genomic mutation regions are employed to determine both copy-number and genetic alterations at the desired regions in the genomic locus. FIG. 3 provides a schematic of different genomic regions in a genomic locus and the baits used to generate an enriched genomic sample that includes a plurality of genomic backbone regions (for the “Genomic Backbone”) and a plurality of genomic mutation regions of interest (for the SNPs, clinical disease-associated regions, and other targeted exons).

The genomic backbone comprises two or three overlapping baits (probe groups 1, 2, and 3 in FIG. 3) located approximately 50 kb apart. In the Clingen regions, the baits are spaced approximately 5-10 kb apart. Average length of the merged bait interval used in copy number calling in the backbone is around 140 bp. Baits in targeted genes in the focused exome are typically tiled across individual exons.

The enriched genomic region may be enriched from an initial genomic sample using any convenient method, e.g., using hybridization to an oligonucleotide probe (or “bait”) or using a ligation-based method. In some embodiments, the genomic region may be enriched by hybridization in solution to one or more biotinylated oligonucleotides (which, in certain cases, may be RNA oligonucleotides) that may be from 20 to 200 nt in length, e.g., 100 to 150 nt in length, to capture regions of interest. In these embodiments, after capture, duplexes containing fragments of genomic DNA that hybridize to the oligonucleotides may be isolated from other fragments using, e.g., streptavidin beads. In other embodiments, the region of interest may be enriched using the method described by Dahl et al., Multiplex amplification enabled by selective circularization of large sets of genomic DNA fragments, Nucleic Acids Res., 2005, 33: e71. In this method, a genomic sample may be fragmented using one or more restriction enzymes and denatured. In this method, a probe library is hybridized to the targeted fragments. Each probe is an oligonucleotide designed to hybridize to both ends of a targeted DNA restriction fragment, thereby guiding the targeted fragments to form circular DNA molecules. The probe also contains a method-specific sequencing motif that is incorporated during circularization. In some cases, the probes are biotinylated and the targeted fragments can be retrieved using streptavidin beads. The circular molecules are then closed by ligation, a very precise reaction that ensures that only perfectly hybridized fragments are circularized. Next, the circular DNA targets are amplified. Other enrichment methods may be described in, e.g., Hedges et al., Comparison of three targeted enrichment strategies on the SOLiD sequencing platform, PLoS One, 2011, 6: e18595; and Shearer et al., Solution-based targeted genomic enrichment for precious DNA samples, BMC Biotechnol., 2012, 12: 20.

The genomic DNA may be isolated from any organism. The organism may be a prokaryote or a eukaryote. In certain cases, the organism may be a plant, e.g., Arabidopsis or maize, or an animal, including reptiles, mammals, birds, fish, and amphibians. In some cases, the initial genomic sample may be isolated from a human or rodent, such as a mouse or a rat. In exemplary embodiments, the initial genomic sample may contain genomic DNA from a mammalian cell, such as, a human, mouse, rat, or monkey cell. Methods of preparing genomic DNA for analysis is routine and known in the art, such as those described by Ausubel, F. M. et al., Short protocols in molecular biology, 3rd ed., 1995, John Wiley & Sons, Inc., New York; and Sambrook, J. et al., Molecular cloning: A laboratory manual, 2^{nd} ed., 1989, Cold Spring Harbor Laboratory Press, Cold Spring Harbor, N.Y. The initial genomic sample may contain genomic DNA or an amplified version thereof (e.g., genomic DNA amplified by a whole genome amplification method using the methods of: Lage et al., Genome Res., 2003, 13: 294-307; Zong et al., Science, 2012, 338:1622-1626; or published U.S. Patent Application Publication no. US20040241658). Fragments may be made by fragmenting a genome using physical methods (e.g., sonication, nebulization, or shearing), chemically, enzymatically (e.g., using a rare-cutting restriction enzyme) or using a transposable element (see, e.g., Caruccio, Methods Mol. Biol., 2011, 733: 241-55; Kaper et al, Proc. Natl. Acad. Sci., 2013, 110: 5552-7; Marine et al, Appl. Environ. Microbiol., 2011, 77: 8071-9; and U.S. Patent Application Publication no. US20100120098).

The sample may be made from cultured cells or cells of a clinical sample, e.g., a tissue biopsy, scrape or lavage or cells of a forensic sample (i.e., cells of a sample collected at a crime scene). In particular embodiments, the nucleic acid sample may be obtained from a biological sample such as cells, tissues, bodily fluids, and stool. Bodily fluids of interest include but are not limited to, blood, serum, plasma, saliva, mucous, phlegm, cerebral spinal fluid, pleural fluid, tears, lactal duct fluid, lymph, sputum, cerebrospinal fluid, synovial fluid, urine, amniotic fluid, and semen. In particular embodiments, a sample may be obtained from a subject, e.g., a human, and it may be processed prior to use in the present method. For example, the nucleic acid may be extracted from the sample prior to use, methods for which are known. In particular embodiments, the genomic sample may be from a formalin fixed paraffin embedded (FFPE) sample.

Depending on which embodiment of the method is implemented, the initial sample (i.e., prior to enrichment) may contain fragments of genomic DNA that are already adaptor-ligated. In other embodiments, the fragments may be ligated to an adaptor after they have been enriched.

In some cases, samples may be pooled. In these embodiments, the fragments may have a molecular barcode to indicate their source. In some embodiments the DNA being analyzed may be derived from a single source (e.g., a single organism, virus, tissue, cell, subject, etc.), whereas in other embodiments, the nucleic acid sample may be a pool of nucleic acids extracted from a plurality of sources (e.g., a pool of nucleic acids from a plurality of organisms, tissues, cells, subjects, etc.), where by “plurality” is meant two or more. As such, in certain embodiments, the sample can contain nucleic acids from 2 or more sources, 3 or more sources, 5 or more sources, 10 or more sources, 50 or more sources, 100 or more sources, 500 or more sources, 1000 or more sources, 5000 or more sources, up to and including about 10,000 or more sources. Molecular barcodes may allow the sequences from different sources to be distinguished after they are analyzed.

After an enriched sample has been obtained, it is, in some embodiments, amplified and then sequenced. In certain embodiments, the fragments are amplified using primers that are compatible with use in, e.g., Illumina's reversible terminator method, Roche's pyrosequencing method (454), Life Technologies' sequencing by ligation (the SOLiD platform) or Life Technologies' Ion Torrent platform. Examples of such methods are described in the following references: Margulies et al., Nature, 2005, 437: 376-80; Ronaghi et al., Analytical Biochemistry, 1996, 242: 84-9; Shendure et al., Science, 2005, 309: 1728-32; Imelfort et al., Brief Bioinform., 2009, 10:609-18; Fox et al., Methods Mol Biol., 2009, 553:79-108; Appleby et al., Methods Mol Biol., 2009, 513:19-39; and Morozova et al., Genomics, 2008, 92:255-64, which are hereby incorporated by reference herein for the general descriptions of the methods and the particular steps of the methods, including all starting products, reagents, and final products for each of the steps.

In one embodiment, the isolated product may be sequenced using nanopore sequencing (e.g. as described in Soni et al., Clin. Chem., 2007, 53: 1996-2001, or as described by Oxford Nanopore Technologies). Nanopore sequencing is a single-molecule sequencing technology whereby a single molecule of DNA is sequenced directly as it passes through a nanopore. A nanopore is a small hole, of the order of 1 nanometer in diameter. Immersion of a nanopore in a conducting fluid and application of a potential (voltage) across it results in a slight electrical current due to conduction of ions through the nanopore. The amount of current which flows is sensitive to the size and shape of the nanopore. As a DNA molecule passes through a nanopore, each nucleotide on the DNA molecule obstructs the nanopore to a different degree, changing the magnitude of the current through the nanopore in different degrees. Thus, this change in the current as the DNA molecule passes through the nanopore represents a reading of the DNA sequence. Nanopore sequencing technology is disclosed in U.S. Pat. Nos. 5,795,782; 6,015,714; 6,627,067; 7,238,485; and 7,258,838; and in U.S. Patent Application nos.: US2006003171 and US20090029477.

In some embodiments, the sequencing may produce, for each enriched region at least 100, at least 1,000, at least 10,000 up to 100,000 or more sequence reads. The length of the sequence reads may vary greatly depending on, for example, the platform used. In some embodiments, the length of sequence reads may be in the region of 30 to 800 bases and, in some cases, may include paired end reads.

Once sequences for the enriched genomic sample and the corresponding reference sample are obtained, all of the sequence reads are assembled to produce a single pile-up that is examined to identify the number of sequence reads across the entire genomic locus (the genomic locus from which the enriched genomic samples are derived) as well as sequence reads that have a nucleotide variation (e.g., a substitution, insertion or deletion) at a particular position. Sequence reads may be assembled using any convenient method, the basic steps of which are described in a variety of publications such as Myers et al., Science, 2000, 287: 2196-204; Batzoglou et al., Genome Research, 2002, 12: 177-89; Dohm et al., Genome Research, 2007, 17: 1697-706; and Boisvert et al., Journal of Computational Biology, 2010, 17: 1519-33; which are all hereby incorporated by reference herein for disclosure of such methods. In some aspects, the sequence reads can be assembled by aligning each read to a reference sequence, such as a reference genome.

The assembled sequence data is analyzed to determine whether the genomic locus has (i) a copy number variation (CNV) and (ii) one or more of: a loss of heterozygosity (LOH, e.g., cnLOH or UPD), a SNP, a base mutation, or an insertion-deletion (indel).

(i) CNV Detection/Determination

The aligned/assembled sequence reads from the enriched genomic sample and the reference sample (either newly generated or a previously analyzed sample from a database) are streamed together and mapped to log ratios in suitable genomic windows using a map-reduce operation. In some embodiments, a pool of reference samples may be used to generate a synthetic reference in the database. The summarization method used aims at capturing the central tendency of read distributions over genomic regions or baits while minimizing the noise introduced due to outliers. Converting read distributions over baits to log ratios over sample and references(s) removes design or experiment specific biases. Different strategies for summarization can be applied to obtain these log ratios. These include normalizing over baits using a suitable central tendency such as mean and median, homogenizing the tendency over the bait region, normalizing sample and reference separately for typical coverage in each sample separately, or taking a suitable sliding window genomic region instead of the whole bait region.

In one embodiment, for detecting translocation and structural variants, rather than taking log ratios of the read counts, the log ratios of secondary split hits for the reads and/or the count of paired reads in the region whose pairs map anomalously can be taken. The information from bait design used in the capture is then used to normalize the log ratios to reduce biases due to sequence of genomic regions targeted and to reduce variability arising from variation in bait characteristics.

The log ratios are then subjected to an un-decimated wavelet transform to detect abrupt changes or break points at various genomic length scales. The breakpoints are combined together and ranked. False discovery rate (FDR) is controlled and the breakpoints passing the FDR control are scored for statistical significance. Such significant intervals are candidates for amplification and deletion variants.

The position of each breakpoint is further refined by examining the regions containing breakpoints at finer resolution.

The methodology, by its multi-scale nature yields the noise estimate as well as baseline variations without any need for correcting these separately. The aberrant intervals are then assigned copy numbers. This is done by first examining the median values at different wavelet scales and determining putative copy numbers for them by doing a robust egression and identifying gaps as well as slope of trend of copy numbers with ratios.

A mixture model is fitted to the log ratios with Gaussian functions centered on the possible assignments. An optimization routine then evaluates the best fit. Each aberration is scored against the model and copy number is assigned using a Bayesian approach. The algorithm then also assigns scores for the summarized log ratios over an exon or any other suitably defined genomic region to determine copy number of such regions.

(ii) Detection of One or More of: A Loss of Heterozygosity (LOH, e.g., cnLOH or UPD), a SNP, a Base Mutation, or an Insertion-Deletion (Indel)

In parallel to the CNV analyses above, an algorithm to identify differences (or mutations) in the sequence reads of the enriched genomic sample are identified. The differences are sometimes referred to as “polymorphisms”, where a polymorphism may be, e.g., single nucleotide polymorphisms (SNPs), “indels” (i.e., insertions and deletions), inversions, re-arrangements or any other source of genome sequence variation. In certain embodiments, the differences (SNP, mutations, and indels) are detected using a SNPPET algorithm, e.g., as described in U.S. Patent Application Publication no. US20150073724 (hereby incorporated by reference herein). This algorithm facilitates calling of low frequency and multi-allelic SNP sites. Each SNP passing the specified quality criteria is then queried against an SNP databases to obtain its known allele frequencies in various populations, heterozygosity rate, and variance (where available).

In some cases, the aligned reads are remapped to the regions of interest and quality values for bases are recalibrated. After such a read mapping the “candidate regions”, i.e., the merged bait regions for baits overlapping, are examined for the presence of mismatches from the reference genome. A statistical model is fit at each base to ascertain whether a seen mismatch is more likely to be a true sequence variant than a sequencing or mapping error. A variety of criteria are applied to remove capture, sequencing, and alignment errors.

The SNPPET algorithm has two basic steps. The first step is a quick search for variants where each locus is evaluated under two models. One model assumes that at the base under consideration, all non-reference alleles are due to sequencing error and the next model considers each non-reference allele to be a true variant. The second step is a careful local search in the neighborhood of the variant. All potential variant combinations are evaluated as haplotypes, and adjusted for additional nearby variant sites.

In some cases and as will be described in greater detail below, graph theory is used to assemble the reads. In particular cases, especially if the steps above have identified multi-alleic loci and multiple variants within a candidate region, assembling the sequence reads may comprise making a directed graph, such as a de Bruijn graph. For example, constructing a de Bruijn graph of sequence reads may involve: collecting overlapping k-mers from the sequencing reads, including subsequences of length k within the reads, in a target region; splitting each k-mer into two overlapping (k−1)-mers; and assigning a vertex or node of the graph to each (k−1)-mer and an edge connecting the two nodes in the graph to the k-mer. Thus each sequence read is represented in the graph as a path through the k-mers, and a potential sequence contig may be represented in the graph by joining multiple paths through the k-mers. The use of de-Bruijn graphs to assemble reads is described in U.S. Pat. No. 8,209,130; U.S. Patent Application Publication nos.: US20110004413, US20110015863, and US20100063742, which are hereby incorporated by reference herein.

In certain cases the directed graph may be a directed weighted graph. In certain aspects, the directed weighted graph is constructed using k-mers of the same length. In certain embodiments, the choice of which edge to select to construct a potential sequence at a node is made without using a cutoff value that is a function of the read coverage at the particular node or edges connected to the node.

A potential sequence is represented in the directed weighted graph by an Euler path. Thus, assembling the sequence reads may further involve finding an Euler path through the directed weighted graph constructed from the sequence reads. Finding an Euler path through the directed weighted graph may comprise finding a minimal de-Bruijn sequence (i.e., cyclic sequence of a given alphabet A with size k for which every possible subsequence of length n in A appears as a sequence of consecutive characters exactly once) in a language with forbidden strings (see, e.g., Moreno et al., Graph-Theoretic Concepts in Computer Science, 2004, 3353:168). In such cases, a minimum de-Bruin sequence may be defined by a spanning subgraph, or tree of the directed weighted graph, using the BEST (de Bruijn, Ehrenfest, Smith and Tutte) theorem (which provides a product formula for the number of Eulerian circuits in directed (oriented) graphs, and relates the number of Eulerian circuits to the number of rooted spanning trees at a given vertex). Determining spanning trees of a directed graph may be achieved by any convenient method (see, e.g., Tarjan et al., Proc FOCS, 1984, 12-20). Representation of the weighted directed graph as a de Bruin sequence with forbidden words leads to an estimate of the maximum number of words possible in the graph and reflects the information entropy of the directed graph. This entropic bound is also a limit on the eigenvalue of the transition matrix of the directed graph. As the bound for information entropy is determined by the directed graph constructed from the sequence reads, any potential variant sequence that cannot be derived, given the set of sequencing reads, from a reference or a another potential variant without exceeding the information entropy bound (i.e., if the eigenvalue for the transition matrix between the potential variant and another variant or reference exceeds the bound established above) will be discarded.

In certain cases, the sequence reads may be anchored on a reference sequence, which will be discussed in greater detail below. In some embodiments the sequence assembly method involves, in each of the sequence reads, demarcating the regions where sequencing is deemed reliable, and each of the assemblies may be anchored using the reference sequence as well as sequences that are local to the reference sequence.

In this method, the sequence assembly step results in a plurality of discrete assemblies, where each assembly corresponds to a potential variant. Each potential variant is defined by a sequence variation that is found in the sequence reads. As such, all of the candidate sequences in a discrete assembly have the same variation. Any one enriched region may be represented by at least 2, at least 5, at least 10, at least 15, at least 20, at least 30, at least 50, at least 100 or more discrete assemblies. The number of sequence reads in each assembly may vary greatly. In several cases, the majority of sequence reads may assemble into one or two assemblies that represent the dominant variants in the sample (depending on whether the original sample from which the genomic DNA was originally obtained is homozygous or heterozygous for a germline difference, e.g., a SNP, in the enriched region). The remaining assemblies may correspond to low frequency variant sequences (e.g., sequences obtained from somatically mutated cells), may be derived from PCR errors, and/or may contain mis-called bases. In certain cases, these assemblies may be represented by fewer sequence reads that contain the variation (e.g., 10 to 1,000 or more, depending on the total number of sequence reads that are obtained).

In the next step of the method, the discrete assemblies are screened to determine which of the potential variants are “true” (i.e., correctly provide the sequence for a molecule in the sample and are not the result of an error in the sequencing reaction or data processing, e.g., a base mis-call) and which candidate molecules are artifacts (i.e., are the result of an error in the sequencing reaction or data processing, e.g., a base mis-call, and not the actual sequence of a molecule in the sample). This step may be done by examining the sequence reads that make up each of the discrete sequence assemblies. In some embodiments, this step may be done by examining a variety of parameters, including read quality, the confidence of a base call, and confidence of an alignment (i.e., whether the sequence has mapped to the right location). Weakly defined candidate molecules (i.e., candidate molecules that are defined by poor sequence reads, candidate molecules in which the sequence variant is represented by a low confidence base call, etc.) can be dissolved, and the sequence can be merged with other alignments. In certain embodiments, the likelihood of each potential variant, given the set of sequencing reads, is assigned using a Hidden Markov model. In some embodiments, this step may comprise examining the quality of a sequence, the number of reads, the quality of base calls and their match to the reference sequence, to provide a score for each of the potential variants.

Once the true potential variants have been identified, the mutations defined by the potential variants can be optionally compared to the known mutations for a reference sequence, where the reference sequence is a sequence from a public or in-house database. In certain embodiments the comparing involves determining whether each of the true potential variants contains a mutation that is known to be associated with the reference sequence. For example, the identity of several thousand cancer-associated mutations in several hundred genes can be found at the Sanger Center's COSMIC database (see also Jung et al., Systematic investigation of cancer-associated somatic point mutations in SNP databases, Nature Biotechnology, 2013, 31: 787-789).

Once differences in the sequences from the enriched genomic sample are identified, LOH detection may proceed if desired. For known heterozygous SNPs with allele frequencies known in the population, the entire sample is assigned to a population using Bayesian scoring against known allele frequencies. If such an assignment is successful, which is measured by the probability of the sample belonging to a population, then allele frequencies from the identified population are used. However, if population assignment fails or is ambiguous, average heterozygosity rates from overall populations are selected and used (e.g., based on sequence data obtained from public or private databases, e.g., the NCBI sequence database). Copy number information obtained for the enriched genomic sample (e.g., as discussed above) is utilized in determining accurate adjustment to allele frequencies.

A sequential Fishers test over “spatial” genomic data is then performed to determine genomic regions enriched in SNPs which have LOH. The test can handle the complex situations arising from the presence of indels and multiple alleles and assigns a score to a genomic region that reflects its enrichment in SNPs that have high likelihood of LOH in the sample. The score is a summarization of (i) p-values for both SNPs being homozygous in a sample given its probability of being a known heterozygous, and (ii) the unexpectedness of seeing it statistically significantly enriched in such SNPs in a genomic region. A threshold on the score determines the boundaries of the region.

In certain embodiments, the LOH analysis includes a determination of whether the LOH is copy neutral, i.e., that the region of LOH is neither increased nor decreased in its copy number in the genomic sample (e.g., for diploid genomes, the LOH region would still have 2 copies).

The methods detailed above may include outputting a report indicating whether the sample comprises a particular genomic alteration, e.g., a CNV, polymorphism, and/or a sequence variant/mutation. The report may further contain available public information about the reference sequence and the genomic alteration. In some cases, the report may indicate the confidence that the genomic alteration is in the sample.

The above-described method may be employed to characterize, classify, differentiate, grade, stage, diagnose, or prognose a condition, e.g., a disease condition, or to predict a response to treatment for a condition. In particular cases, the method may be used to investigate a cancerous condition or another mammalian disease, including but not limited to, leukemia, breast carcinoma, prostate cancer, Alzheimer's disease, Parkinson's disease, epilepsy, amyotrophic lateral sclerosis, multiple sclerosis, stroke, autism, mental retardation, and developmental disorders. Many copy number changes and/or nucleotide polymorphisms are associated with and are thought to be a factor in producing these disorders. Knowing the type and the location of the such genetic alterations may greatly aid the diagnosis, prognosis, and understanding of various mammalian diseases. Other applications of the disclosed methods and systems are envisioned, including, for example, forensics, epidemiology, and other areas where genomic alteration detection is of use.

In some embodiments, a biological sample, e.g., a biopsy, may be obtained from a patient, and the sample may be analyzed using the disclosed method. In these embodiments, the method may be employed to detect an oncogenic genetic alteration (which may be a somatic mutation) in, e.g., PIK3CA, NRAS, KRAS, JAK2, HRAS, FGFR3, FGFR1, EGFR, CDK4, BRAF, RET, PGDFRA, KIT or ERBB2, which mutation may be associated with breast cancer, melanoma, renal cancer, endometrial cancer, ovarian cancer, pancreatic cancer, leukemia, colorectal cancer, prostate cancer, mesothelioma, glioma, medullobastoma, polycythemia, lymphoma, sarcoma or multiple myeloma (see, e.g., Chial, Proto-oncogenes to oncogenes to cancer, Nature Education, 2008, 1:1).

Because the genomic alteration in a genomic locus may have a direct association with cancer, the subject method may be employed to diagnose patients with cancer or a pre-cancerous condition (e.g., adenoma etc.), alone, or in combination with other clinical techniques (e.g., a physical examination, such as, a colonoscopy or mammogram) or molecular techniques (e.g., immunohistochemical analysis). For example, results obtained from the subject assay may be combined with other information, e.g., information regarding the methylation status of other loci, cytogenetic information, gene expression information or information about the length of telomeres, to provide an overall diagnosis of cancer or other diseases.

In one embodiment, a sample may be collected from a patient at a first location, e.g., in a clinical setting such as in a hospital or at a doctor's office, and the sample may be forwarded to a second location, e.g., a laboratory where it is processed and the above-described method is performed to generate a report. A “report” as described herein, is an electronic or tangible document which includes report elements that provide test results that may include a Ct value, or Cp value, or the like that indicates the presence of CNV and/or mutant copies of the genomic locus in the sample. Once generated, the report may be forwarded to another location (which may the same location as the first location), where it may be interpreted by a health professional (e.g., a clinician, a laboratory technician, or a physician such as an oncologist, surgeon, pathologist) as part of a clinical diagnosis.

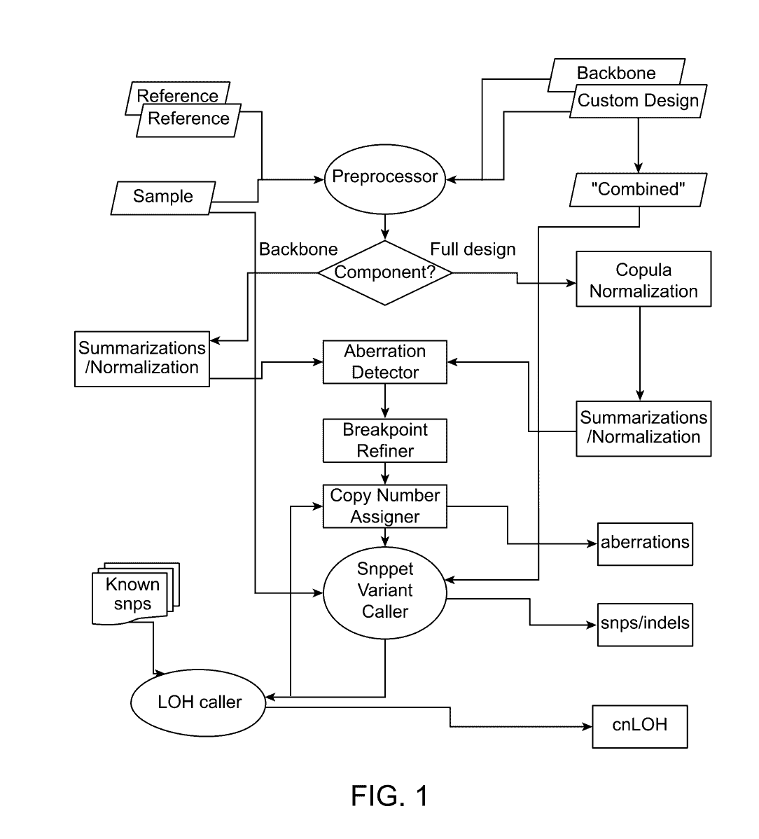

Aspects of the disclosed methods are shown in the flow chart of FIG. 1. It is noted that in certain embodiments of the present disclosure, only a subset of the steps in FIG. 1 are performed.

In the Preprocessor step, the information from backbone and the custom part of the design (Custom Design) are used to summarize aligned read counts into a log ratio between test sample and one or more reference sequences.

To detect copy number variations, the reads overlapping with the merged bait regions are collected. Only the reads that pass vendor QC and alignment quality criteria are kept. These criteria include mapping quality thresholds, number of multiple hits, matched pairs and a minimum specified read count in the reference. The counts are adjusted for differing number of total reads in sample and reference and for non-uniformity in capture. A log ratio of the normalized counts is then taken.

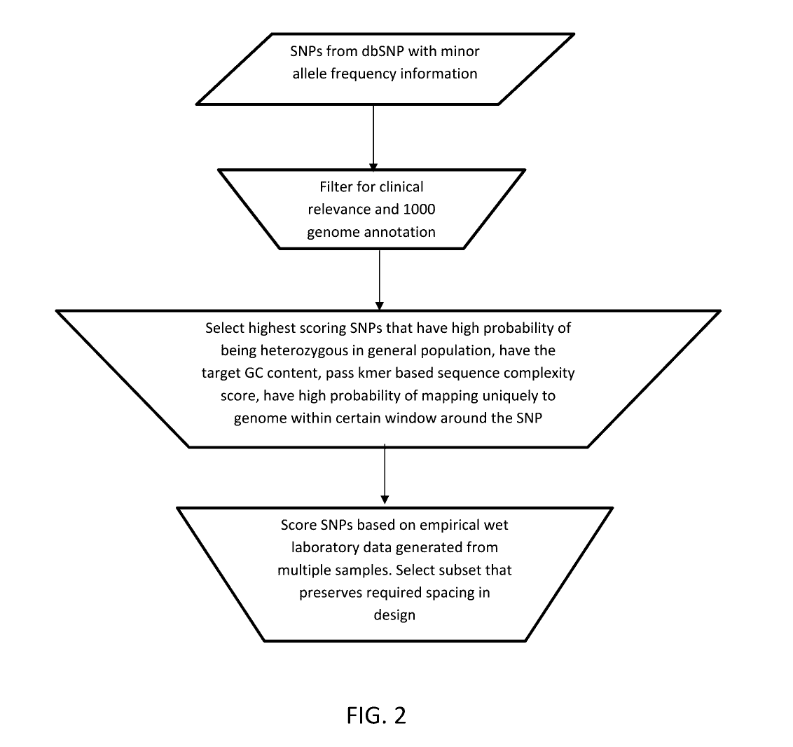

The flow chart in FIG. 2 shows an example of one embodiment of a selection process for baits specific for SNPs in a genome of interest. This representative selection process involves utilizing average heterozygosity and allele frequency information compiled on SNPs for a particular genome build. SNPs that have been marked for clinical relevance are chosen and evaluated further for a desired range of genomic properties. The bait filtering is done based on the percentage GC content, mappability, entropy, and dust scores. The mappability is obtained from averaging known mappability scores from UCSC genome tracks over the bait region. The entropy and dust scores control the sequence complexity and variations due to repeat sequences.

In the Component step, the two components of the same design, i.e., the backbone and the custom part in design, are adjusted so that they are on the same footing. If the read distributions in the complement part are the same as that in the backbone part, then the complement is just collected along with the backbone. If the distributions are different, then the two distributions are treated as a Gaussian Copula. To fulfill the requirement of analysis of such a copula, the custom part is corrected by using a ratio transformation, such as Greary Hinkley transform, to transform it to a Gaussian form. Standard methods for handling Gaussian Copulas can then be used and then rescaled to have equivalent distribution to the backbone. Examples of Gaussian Copulas are described in: Roger B. Nelsen, “An Introduction to Copulas”, Springer, 1999. (ISBN 978-O-387-98623-4); Piotr Jaworski, Fabrizio Durante, Wolfgang Karl Härdle, Tomasz Rychlik (eds), “Copula Theory and Its Applications”, Lecture Notes in Statistics, Springer, 2010 (ISBN 978-3-642-12464-8); Abe Sklar “Random variables, distribution functions, and copulas—a personal look backward and forward”, In Rüschendorf, L., Schweizer, B. and Taylor. M. (eds), “Distributions With Fixed Marginals & Related Topics”. Lecture Notes, Monograph Series Number 28, 1997 (ISBN 978-O-940600-40-9); each of which are hereby incorporated by reference herein. The log ratio values are further corrected for GC bias variability by subtracting out any sample-wide baseline trends by performing a robust regression with GC content and subtracting the baseline. The same bias correction is performed with respect to the mappability.

Next, the normalized log ratios are analyzed by an Aberration Detector module. The Aberration Detector analyzes the log ratio signal in a succession of resolution levels in powers of two and detects abrupt variation at each level. These candidate breakpoints are then examined across the level and adjusted for level spacings. This is performed by using an undecimated discrete wavelet transform algorithm. The algorithm also yields a noise estimate of the data at the lowest level and any residual baselines at the highest one. After combining candidate breakpoints across levels the breaks are scored for significance and ranked by their decreasing levels of significance. A multiple testing correction procedure then selects the most significant breakpoints and a supplied false discover rate (FDR) threshold is used to control the error. User-defined filters on the number of points allowed in the aberrant region, the minimum average log ratios, and minimum aberration size are utilized to filter out regions which don't meet these criteria.

Next, the Breakpoint Refiner examines significant breakpoints in more minute detail by re-examining reads in the neighborhood of these locations. The counts are re-valuated at a finer scale around these regions to further refine the location of the breakpoints.

In the next step, the breakpoints and their wavelet transforms are used to assign copy number to an aberration region defined by successive breakpoints (the Copy Number Assigner). Each coefficient contains information about a particular scale and the gaps between the coefficients are used to find indications of copy numbers present. A mixture of Gaussian, defined by the mass under each band of coefficients, the noise level and the median log ratios in each band is used to find copy number of each region as expectation value of copy number given the mixture.

The SNPPET caller (described above) is then used to call SNPS and indels from the test sample alone. The complete design, i.e. the backbone and the complement is used for this purpose with an option of padding each merged bait region (see, e.g., US Patent Application Publication no. US20150073724, hereby incorporated by reference herein). The SNPPET algorithm has two basic steps. The first step is a quick search for variants where each locus is evaluated under two models. One model assumes that at the base under consideration, all non-reference alleles are due to sequencing error and the next model considers each non-reference allele to be a true variant. The second step is a careful local search in the neighborhood of the variant. All potential variant combinations are evaluated as haplotypes, and adjusted for additional nearby variant sites. In parallel, the SNPS covered by design are queried from the databases along with their known population frequencies in databases. The status of each SNP called or covered by design is ascertained. A Bayesian procedure is used to try and assign the sample to known population(s). If such an assignment is successful with high probability, the known allele frequencies from the corresponding populations are used in subsequent analysis. Otherwise the average heterozygosity rate and its associated standard error are used. Each SNP locus is then assigned a probability for loss of heterozygosity and the region is scored for loss of heterozygosity by using a sequential 2×2 test between estimated expectation value of allele frequencies against the null of heterozygosity with the known weights. The regions are continued till the score falls below a specified significance threshold or a minimum number of heterozygous loci have been seen.

The above-described method can be implemented on a computer. In certain embodiments, a general-purpose computer can be configured to a functional arrangement for the methods and programs disclosed herein. The hardware architecture of such a computer is well known by a person skilled in the art, and can comprise hardware components including one or more processors (CPU), a random-access memory (RAM), a read-only memory (ROM), an internal or external data storage medium (e.g., hard disk drive). A computer system can also comprise one or more graphic boards for processing and outputting graphical information to display means. The above components can be suitably interconnected via a bus inside the computer. The computer can further comprise suitable interfaces for communicating with general-purpose external components such as a monitor, keyboard, mouse, network, etc. In some embodiments, the computer can be capable of parallel processing or can be part of a network configured for parallel or distributive computing to increase the processing power for the present methods and programs. In some embodiments, the program code read out from the storage medium can be written into a memory provided in an expanded board inserted in the computer, or an expanded unit connected to the computer, and a CPU or the like provided in the expanded board or expanded unit can actually perform a part or all of the operations according to the instructions of the program code, so as to accomplish the functions described below. In other embodiments, the method can be performed using a cloud computing system. In these embodiments, the data files and the programming can be exported to a cloud computer, which runs the program, and returns an output to the user.

A system can in certain embodiments comprise a computer that includes: a) a central processing unit; b) a main non-volatile storage drive, which can include one or more hard drives, for storing software and data, where the storage drive is controlled by disk controller; c) a system memory, e.g., high speed random-access memory (RAM), for storing system control programs, data, and application programs, including programs and data loaded from non-volatile storage drive; system memory can also include read-only memory (ROM); d) a user interface, including one or more input or output devices, such as a mouse, a keypad, and a display; e) an optional network interface card for connecting to any wired or wireless communication network, e.g., a printer; and f) an internal bus for interconnecting the aforementioned elements of the system.

The memory of a computer system can be any device that can store information for retrieval by a processor, and can include magnetic or optical devices, or solid state memory devices (such as volatile or non-volatile RAM). A memory or memory unit can have more than one physical memory device of the same or different types (for example, a memory can have multiple memory devices such as multiple drives, cards, or multiple solid state memory devices or some combination of the same). With respect to computer readable media, “permanent memory” refers to memory that is permanent. Permanent memory is not erased by termination of the electrical supply to a computer or processor. Computer hard-drive ROM (i.e., ROM not used as virtual memory), CD-ROM, floppy disk and DVD are all examples of permanent memory. Random Access Memory (RAM) is an example of non-permanent (i.e., volatile) memory. A file in permanent memory can be editable and re-writable.

Operation of the computer is controlled primarily by an operating system, which is executed by the central processing unit. The operating system can be stored in a system memory. In some embodiments, the operating system includes a file system. In addition to an operating system, one possible implementation of the system memory includes a variety of programming files and data files for implementing the method described below. In certain cases, the programming can contain a program, where the program can be composed of various modules, and a user interface module that permits a user to manually select or change the inputs to or the parameters used by the program. The data files can include various inputs for the program.

In certain embodiments, instructions in accordance with the method described herein can be coded onto a computer-readable medium in the form of “programming,” where the term “computer readable medium” as used herein refers to any storage or transmission medium that participates in providing instructions and/or data to a computer for execution and/or processing. Examples of storage media include a floppy disk, hard disk, optical disk, magneto-optical disk, CD-ROM, CD-R, magnetic tape, non-volatile memory card, ROM, DVD-ROM, Blue-ray disk, solid state disk, and network attached storage (NAS), whether or not such devices are internal or external to the computer. A file containing information can be “stored” on computer readable medium, where “storing” means recording information such that it is accessible and retrievable at a later date by a computer.

The computer-implemented method described herein can be executed using programs that can be written in one or more of any number of computer programming languages. Such languages include, for example, Java (Sun Microsystems, Inc., Santa Clara, Calif.), Visual Basic (Microsoft Corp., Redmond, Wash.), and C++(AT&T Corp., Bedminster, N.J.), as well as any many others.

In any embodiment, data can be forwarded to a “remote location,” where “remote location,” means a location other than the location at which the program is executed. For example, a remote location could be another location (e.g., office, lab, etc.) in the same city, another location in a different city, another location in a different state, another location in a different country, etc. As such, when one item is indicated as being “remote” from another, what is meant is that the two items can be in the same room but separated, or at least in different rooms or different buildings, and can be at least one mile, ten miles, or at least one hundred miles apart. “Communicating” information refers to transmitting the data representing that information as electrical signals over a suitable communication channel (e.g., a private or public network). “Forwarding” an item refers to any means of getting that item from one location to the next, whether by physically transporting that item or otherwise (where that is possible) and includes, at least in the case of data, physically transporting a medium carrying the data or communicating the data. Examples of communicating media include radio or infra-red transmission channels as well as a network connection to another computer or networked device, and the internet or including email transmissions and information recorded on websites and the like.

Some embodiments include implementation on a single computer, or across a network of computers, or across networks of networks of computers, for example, across a network cloud, across a local area network, on hand-held computer devices, etc. Embodiments include implementation on computer program(s) performing one or more of the steps described herein. Such computer programs execute one or more of the steps described herein. Embodiments of the invention include various data structures, categories, and modifiers described herein, encoded on computer-readable medium(s) and transmissible over communications network(s).

Software, web, Internet, cloud, or other storage and computer network implementations of the present invention could be accomplished with standard programming techniques to accomplish the various database searching, modifying, correlating, comparing, deciding, signaling, scoring, surveilling, or ranking steps.

Certain embodiments of the present disclosure include systems for detecting genomic alterations, where the systems include a database configured to store reference sequence reads for one or more reference genomes and a computing device that is configured to perform the genomic sequence analysis methods described above. Thus, in certain embodiments, the computing device is configured to: (a) obtain a plurality of sequence reads from an enriched genomic sample that includes a plurality of genomic backbone regions and a plurality of genomic mutation regions of interest in a genomic locus of a subject; (b) obtain from the database a plurality of reference sequence reads from corresponding genomic backbone regions and genomic mutation regions of at least one reference genome; (c) assemble the plurality of sequence reads from the enriched genomic sample and the at least one reference genomic sample; and (d) determine, based on computational analysis of the assembly, whether the genomic locus has a copy number variation (CNV) or a sequence variation, or a CNV and a sequence variation.

Additional aspects of the present disclosure include computer readable media comprising: (a) a code sequence to obtain a plurality of sequence reads from an enriched genomic sample that includes a plurality of genomic backbone regions and a plurality of genomic mutation regions of interest in a genomic locus of a subject; (b) a code sequence to obtain a plurality of sequence reads from corresponding genomic backbone regions and genomic mutation regions of at least one reference genomic sample; (c) a code sequence to assemble the plurality of sequence reads from the enriched genomic sample and the at least one reference genomic sample; and (d) a code sequence to determine, based on computational analysis of the assembly, whether the genomic locus has a copy number variation (CNV) or a sequence variation, or a CNV and a sequence variation.

### EXPERIMENTAL

### A. Methods

(i) Target Enrichment Panel Design

Target enrichment baits (or probes) were designed and included (1) a set of backbone baits for genome-wide copy number change detection by comparing an experimental sample to a known reference sample, (2) a set of baits that target genomic regions with high minor allele frequency SNPs allowing for the detection of cnLOH, and (3) a set of baits that target specific regions of interest for the detection of mutations and indels. FIG. 3 shows a 28 Mb design that includes baits (12 Mb) for a functional copy number resolution of 300 kb and cnLOH resolution of 5 Mb in the genome-wide backbone, and a higher 25-50 kb resolution in disease-associated ClinGen regions. It also includes all content (16 Mb) from the Agilent SureSelect Focused Exome Panel targeting disease-associated genes. The NV Backbone+Custom Panel allows for the addition of the genome-wide backbone to any custom target gene panel, up to 12 Mb.

(ii) Sample Preparation

Six DNA samples obtained from the Coriell Cell Repository (www.coriell.org), NA03997, NA11419, NA08254, NA04592, NA02948, and NA20409, were processed by following the Agilent SureSelectXT Target Enrichment System for Illumina Paired-End Sequencing Library Version B.1 using 200 ng DNA per sample and 10 post-capture PCR cycles. Agilent Male and Female human reference DNA samples were processed in parallel and were used as controls in the data analysis. Each captured library was loaded on the Illumina Hiseq2500 2×100 bp platform for sequencing. Adequate depth and coverage was achieved with 7 Gb of sequencing per sample.

(iii) Data Analysis

The raw image files were processed by the Illumina base calling software with default parameters and FASTQ files were generated. The FASTQ files were then imported in the Agilent SureCall software v3.0. After removal of the adaptor sequences, the reads were aligned to the genome using the BWA alignment algorithm incorporated in SureCall. Copy number changes are detected by comparing an experimental sample to a known reference sample (see FIG. 4, described below). First, a summarization method is applied to capture the central tendency of read distributions over genomic regions covered by baits with the goal of minimizing the noise introduced due to outliers. Aberrations are called on log ratios that are generated by dividing read depth of the sample over a reference at each bait interval. The log ratios are then subjected to an undecimated wavelet transform to detect abrupt changes or break points. The transformed log ratios are then analyzed at various genomic length scales. After combining and ranking the resulting breakpoints, a false discovery rate step is used to only select those that pass a certain threshold for statistical significance. The significant intervals are then further considered as candidates for amplifications and deletions by examining them at a finer resolution.

FIG. 4 provides a graph for determination of copy number changes in Agilent SureCall software. Log₂ ratios of the sequencing read-depth of the experimental enriched genomic sample versus the sequencing read-depth of the reference (or control) are plotted along the chromosome. No copy number change corresponds to a log₂ ratio of 0, a one copy loss corresponds to a log₂ ratio of -1, a one copy gain corresponds to a log₂ ratio of 0.58.

The SNP calling algorithm SNPPET was used to call point mutations and indels (see, e.g., US Patent Application Publication no. US 20150073724, hereby incorporated by reference herein). The SNPPET algorithm has two basic steps. The first step is a quick search for variants where each locus is evaluated under two models. One model assumes that at the base under consideration, all non-reference alleles are due to sequencing error and the next model considers each non-reference allele to be a true variant. The second step is a careful local search in the neighborhood of the variant. All potential variant combinations are evaluated as haplotypes, and adjusted for additional nearby variant sites.

The high minor allele frequency SNPs covered in the backbone are used to determine cnLOH. Regions of cnLOH or UPD are located by identifying genomic regions with a statistically significant scarcity of heterozygous SNP calls. The LOH algorithm first attempts to assign the sample to a known population with 99% confidence using the allele frequencies determined at the available SNP locations. In cases where a sample cannot be assigned to a known population, average heterozygosity rates available from UCSC are used instead. Then a sequential Fisher's test is used to score genomic regions that are enriched in SNPs that have lost heterozygosity. The final LOH score takes into account the presence of indels and multiple alleles that might be present at the candidate SNP locations. A more detailed description of the algorithms can be found in the SureCall help system.

### B. Results

(i) QC Data

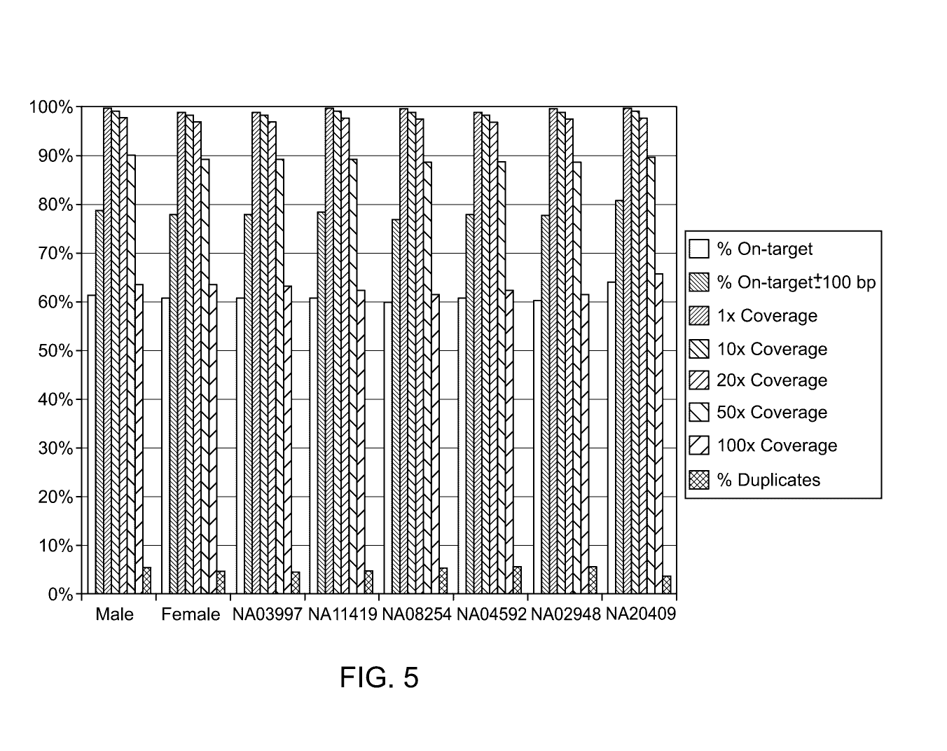

For all eight samples the percentage of reads on target ±100 bp was higher than 75% and the number of duplicate reads was very low (see FIG. 5). This is similar to other SureSelect target enrichment kits such as the Human All Exon V5 kit. The coverage was high with more than 95% of the bases with at least 20 reads. As expected, the number of bases with at least 50 or 100 reads was significantly lower. When increasing the amount of sequencing from 7 Gb to 10 Gb, the number of bases with at least 50 reads was higher than 90% (data not shown).

(ii) Detection of Large CNVs

Expected whole chromosome copy number changes in several samples with known aberrations were detected. Trisomy 13 was detected in Coriell sample NA02948 with karyotype 47, XY,+13. The measured average log₂ ratio for the entire chromosome was close to the expected log₂ ratio value of 0.58 for a single copy gain (data not shown).

(iii) Comparison with aCGH

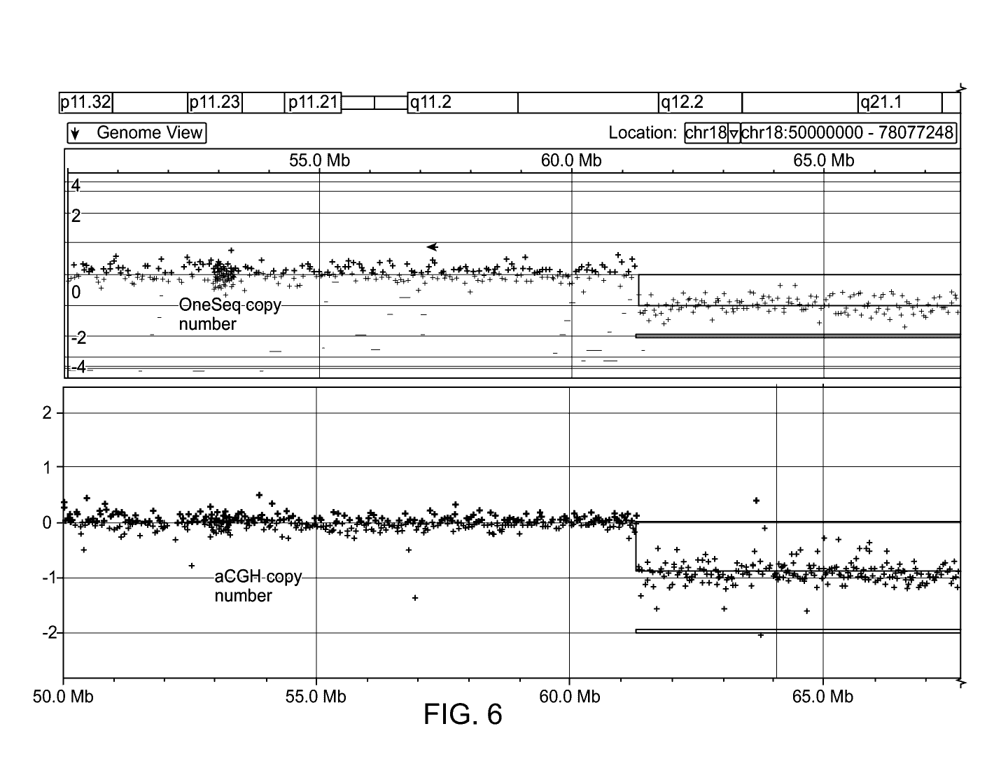

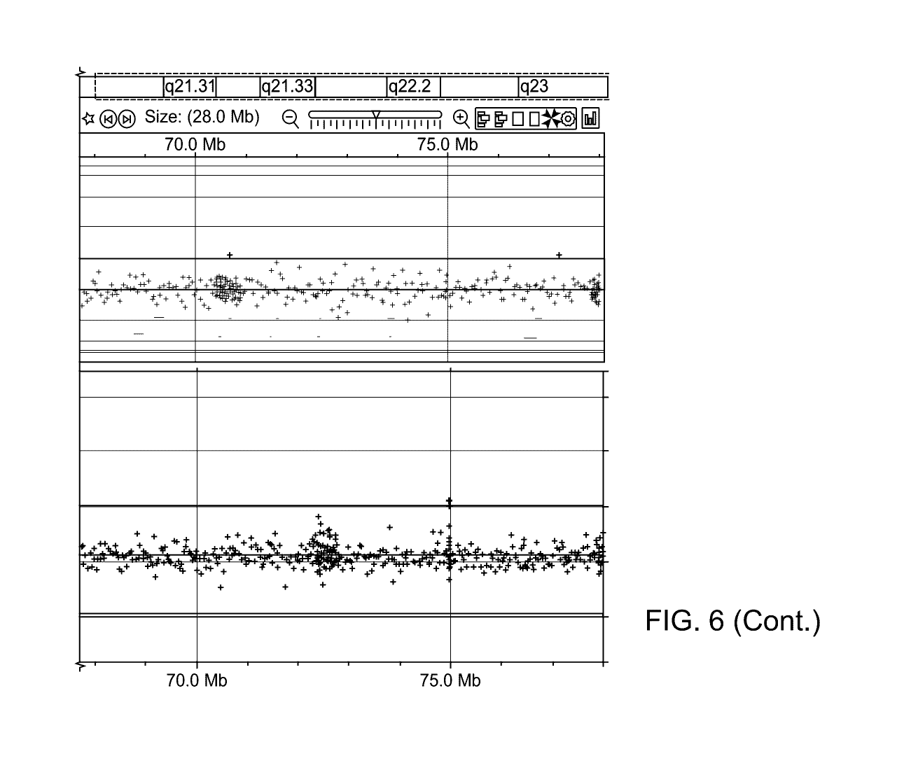

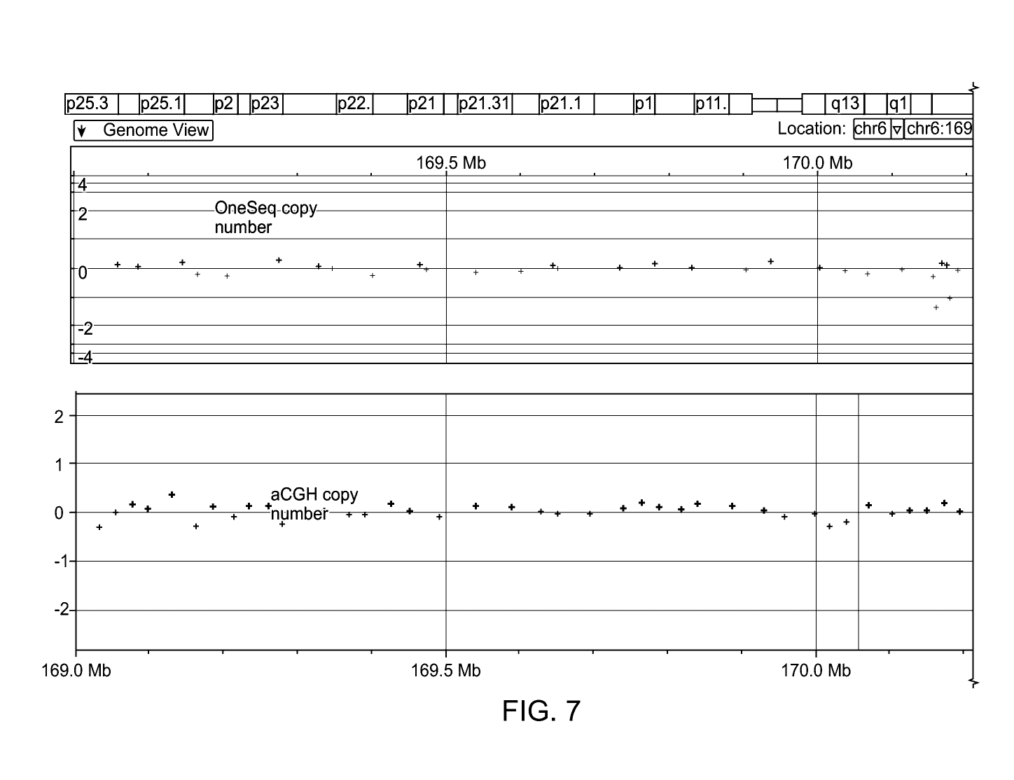

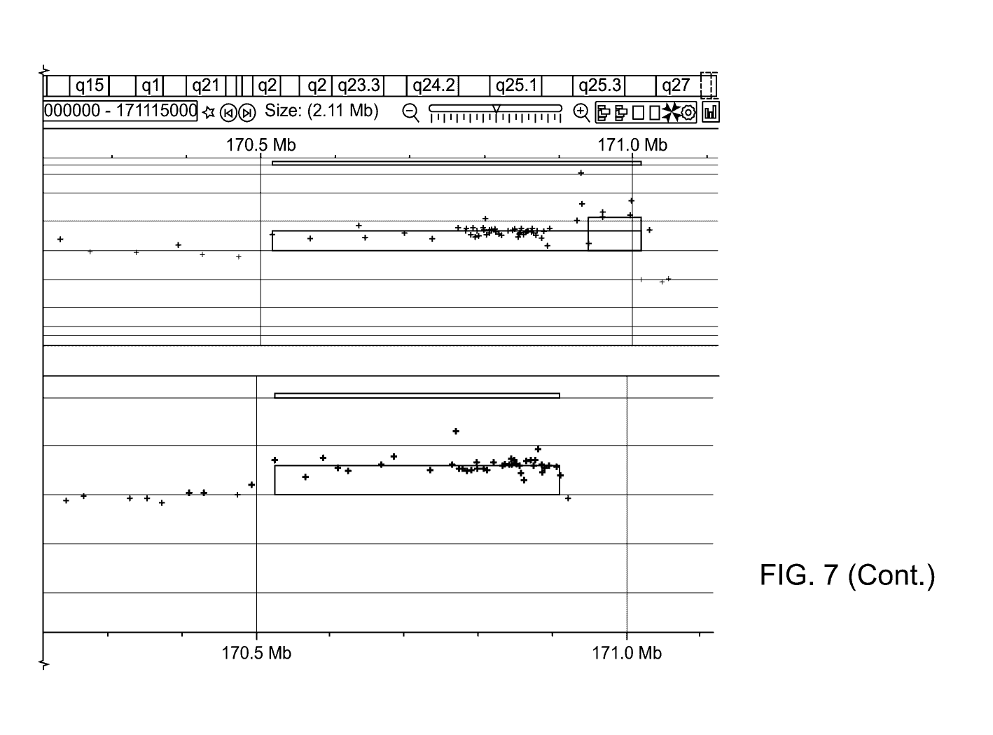

Copy number profiles generated as described above were compared with those obtained by aCGH. The CGH data was generated on the Agilent CGH+SNP 4×180K microarrays. Table 1 shows a comparison of the CNV calls larger than 150 kb obtained by both methods for Coriell sample NA08254. The same eight CNVs could be detected with both methods. The genomic coordinates of the start and stop of the aberrations were not identical due to differences between the aCGH probe and the baits used here, resulting in minor differences in aberration sizes. FIGS. 6 and 7 show the comparison data for a 13 Mb deletion on chromosome 13 and a 370 kb deletion.

TABLE 1
CNVs larger than 150 kb detected in Coriell sample NA08254 by
aCGH and in the present experiment sorted from largest to smallest.

| | | aCGH | Present | Present |
|---|---|---|---|---|
| | Aberration | aberration | expermiment | expriment |
| Chromosome | size type | [kb] | aberration size [kb] | avg log₂ratio |
| chr13 | del | 12427 | 13335 | −0.89 |
| chr15 | del | 2240 | 1667 | −0.37 |
| chr16 | del | 772 | 863 | −0.43 |
| chr14 | amp | 987 | 544 | 0.54 |
| chr6 | amp | 370 | 372 | 0.61 |
| chr2 | del | 828 | 307 | −0.46 |
| chr17 | amp | 163 | 201 | 0.49 |
| chr22 | amp | 172 | 191 | 3.00 |

(iv) Detection of cnLOH

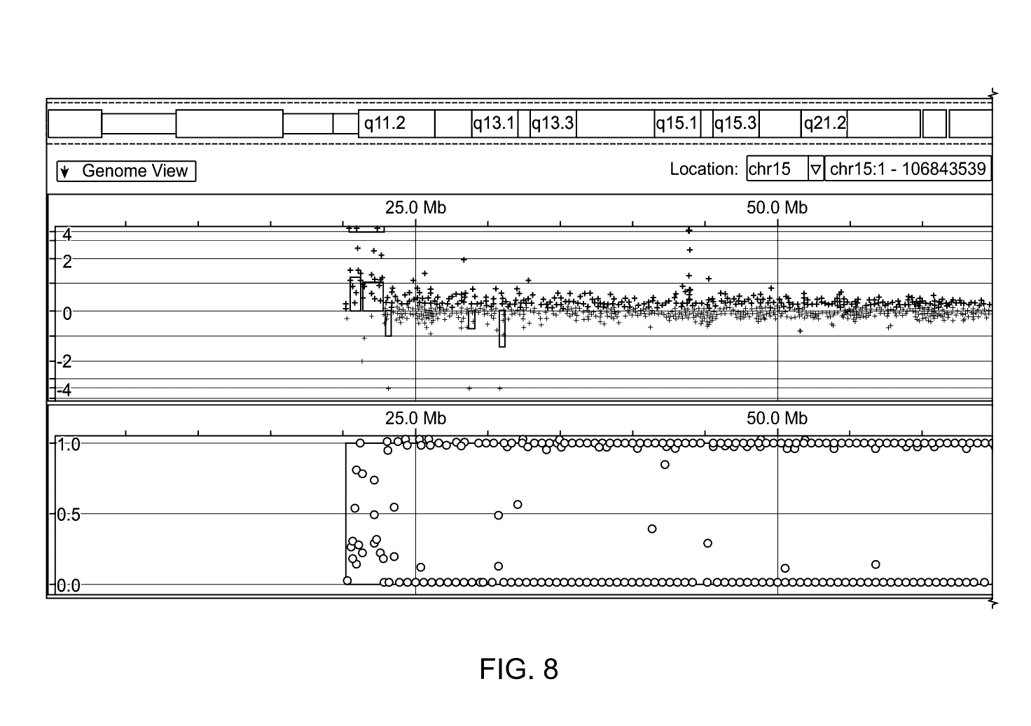

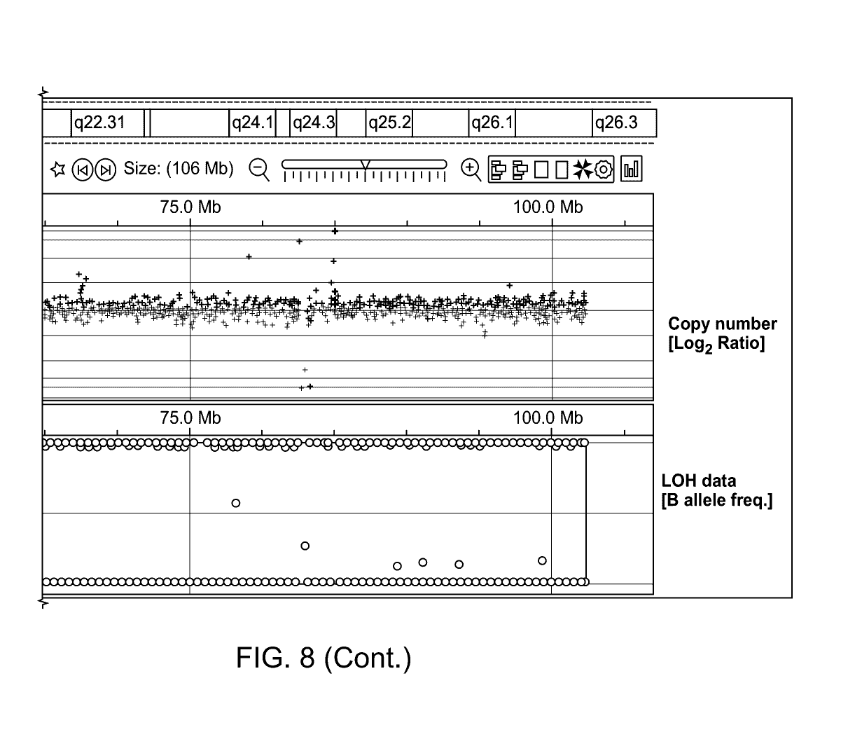

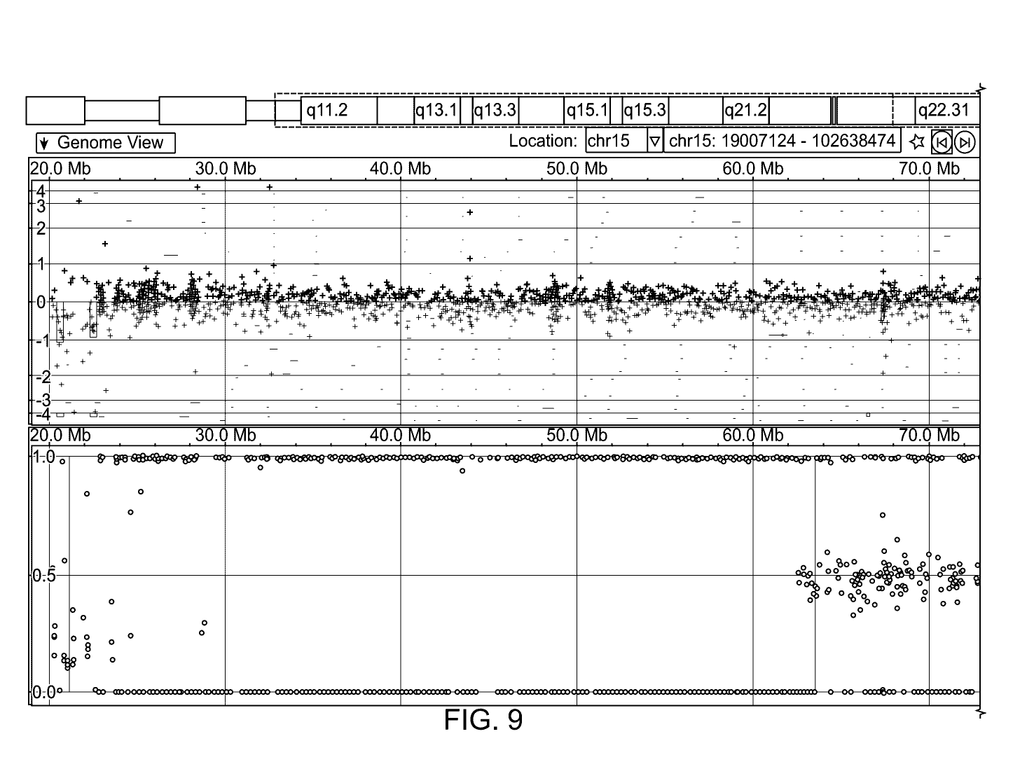

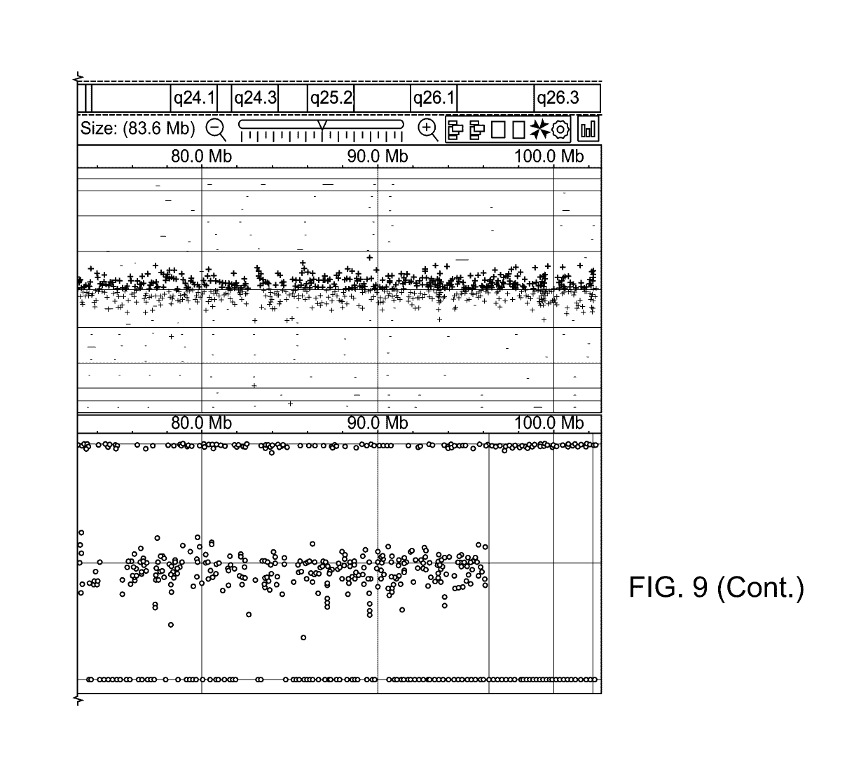

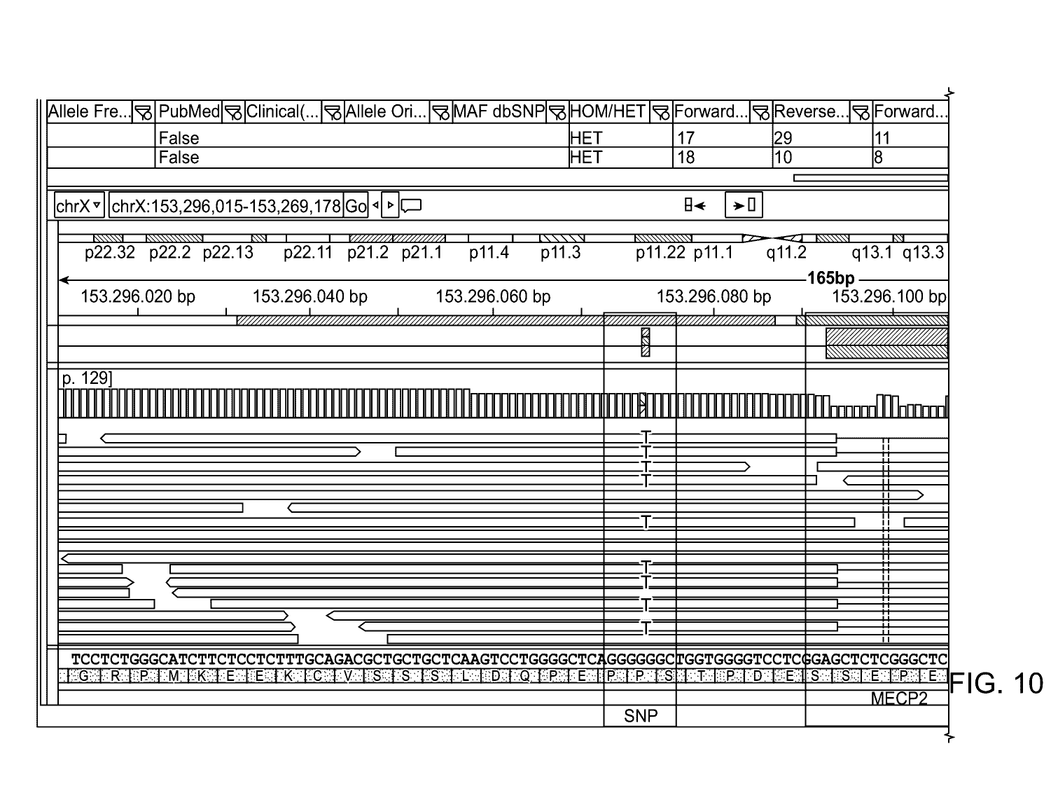

The detection of UPD 15 (uniparental disomy) in Coriell sample NA20409 with complete paternal UPD associated with Angelman Syndrome is shown in FIG. 8. This UPD call was made with high confidence because the B-allele frequency of almost all SNPs was 0% or 100% and virtually no heterozygous SNPs could be detected across the entire chromosome.

(v) Detection of Mutations and Indels

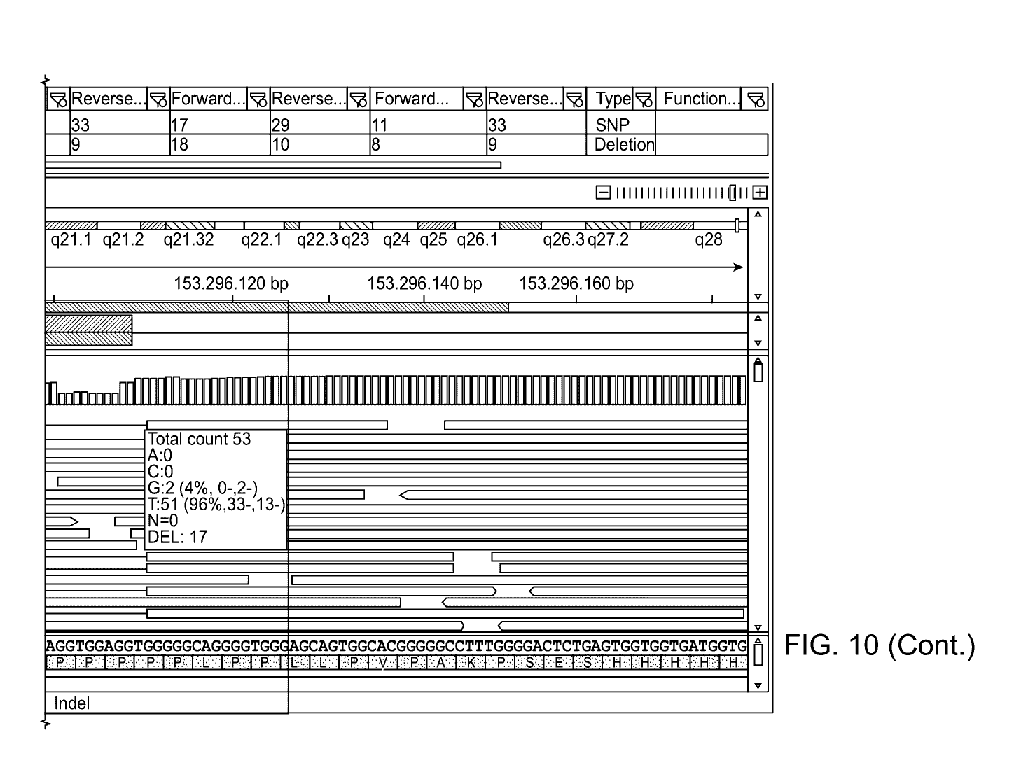

The high read depth allowed for the detection of single point mutations and indels in the targeted regions of interest in all Coriell samples. FIG. 10 shows an example of 26-bp indel in Coriell sample NA16382 known to carry a 26 base pair deletion beginning at nucleotide 1160 of the gene encoding methyl-CpG binding protein 2 (MECP2). A SNP was also identified. In FIG. 10, the sequence reads aligned to the MECP2 gene are shown as block arrows, the indel is indicated by dashed lines and marked by INDEL (this region is split between FIG. 10 and FIG. 10 (Cont.)), and the SNP is indicated with a ‘T’ over each sequence read in which it occurred and is noted at the bottom of the figure. This figure shows that we can identify two events (indel and SNP) in the same region simultaneously.

### C. Conclusion

These experiments demonstrate the effectiveness of the disclosed methods in simultaneously detecting genome-wide copy number variations (CNVs), translocations, LOH (including copy neutral LOH), indels, and gene mutations in one comprehensive assay. Copy number changes from as small as 150 kb to whole chromosome triploidy, stretches of cnLOH, indels, and single base pair mutations, can be detected. The methods disclosed herein do not require that the entire genome be sequenced (as in WGS), but rather rely on the enrichment of genomic backbone regions as well as target specific regions of interest (e.g., sites of SNPs or specific genes/exons of clinical relevance). This approach offers the convergence of existing genetic technologies while maintaining cost-effectiveness and high throughput, thus providing a robust single platform solution for clinical research of a broad range of abnormalities from single gene mutations to aneuploidy.

All publications and patent applications cited in this specification are hereby incorporated by reference herein as if each individual publication or patent application were specifically and individually indicated to be incorporated by reference. The citation of any publication is for its disclosure prior to the filing date and should not be construed as an admission that the present invention is not entitled to antedate such publication by virtue of prior invention.

What is claimed is:

1. A method for detecting genomic alterations, comprising:
(a) obtaining a plurality of sequence reads from an enriched genomic sample that includes a plurality of genomic backbone regions and a plurality of genomic mutation regions of interest in a genomic locus of a subject;
(b) obtaining a plurality of sequence reads from corresponding genomic backbone regions and genomic mutation regions of at least one reference genomic sample;
(c) assembling the plurality of sequence reads from the enriched genomic sample and the at least one reference genomic sample; and
(d) determining, based on computational analysis of the assembly, whether the genomic locus has a copy number variation (CNV) or a sequence variation, or a CNV and a sequence variation.

2. The method of claim 1, wherein determining whether the genomic locus has a sequence variation comprises:
(i) identifying genetic differences in the plurality of sequence reads of the enriched genomic sample as compared to the sequence reads from the enriched genomic sample; and
(ii) determining which of the potential variants are true and which of the potential variants are artifacts by examining the sequence reads that make up each of the discrete sequence assemblies.

3. The method of claim 2, wherein determining whether the genomic locus has a sequence variation comprises using a SNPPET algorithm.

4. The method of claim 1, wherein determining whether the genomic locus comprises a sequence variation further comprises determining whether the genomic locus has a loss of heterozygosity (LOH), comprising:
(i) identifying genetic differences in the sequence reads of the enriched genomic sample as compared to the sequence reads from the at least one reference genomic sample; and
(ii) comparing the identified genetic differences to known frequencies of genetic differences in a population to identify a genomic region in the first genomic locus that has an LOH.

5. The method of claim 1, wherein determining whether the genomic locus has a CNV comprises:
(i) comparing the log ratios of the number of sequence reads from the enriched genomic sample to the number of sequence reads of the at least one reference genomic sample across the first genomic locus; and
(ii) determining a genomic location of one or more break points in the log ratios and selecting one or more of the break points that are statistically significant to detect regions in the first genomic locus that have a CNV.

6. The method of claim 1, wherein determining whether the genomic locus has a sequence variation comprises:
(i) processing sequence reads from two different regions of the enriched genome which differ in at least one statistical property or characteristic to make them amenable to analysis.

7. The method of claim 1, wherein the genomic mutation regions of interest comprise high minor allele frequency single polynucleotide polymorphic sites.

8. The method of claim 1, further comprising (e) outputting a report indicating whether the enriched genomic sample comprises a CNV or a sequence variation, or a CNV and a sequence variation.

9. The method of claim 8, wherein the report indicates whether the enriched genomic sample contains a mutation and provides publicly available information about the reference sequence.

10. The method of claim 1, wherein one or more of the genomic mutation regions are associated with cancer.

11. The method of claim 1, wherein the enriched genomic sample is from a human.

12. The method of claim 1, wherein the assembling uses graph theory.

13. The method of claim 11, wherein the assembling is done using a minimal de-Bruijn sequence.

14. The method of claim 11, wherein the assembling is done using a BEST theorem.

15. The method of claim 1, wherein the enriched genomic sample is obtained using baits designed to target locations in the genome based on known SNP allelic frequency and estimated properties of the genomic regions of interest in the reference genome.

16. A system for detecting genomic alterations comprising:
a database configured to store reference sequence reads for one or more reference genomes; and
a computing device configured to:
(a) obtain a plurality of sequence reads from an enriched genomic sample that includes a plurality of genomic backbone regions and a plurality of genomic mutation regions of interest in a genomic locus of a subject;
(b) obtain from the database a plurality of reference sequence reads from corresponding genomic backbone regions and genomic mutation regions of at least one reference genome;
(c) assemble the plurality of sequence reads from the enriched genomic sample and the at least one reference genomic sample; and
(d) determine, based on computational analysis of the assembly, whether the genomic locus has a copy number variation (CNV) or a sequence variation, or a CNV and a sequence variation.

17. A computer readable medium comprising:
(a) a code sequence to obtain a plurality of sequence reads from an enriched genomic sample that includes a plurality of genomic backbone regions and a plurality of genomic mutation regions of interest in a genomic locus of a subject;
(b) a code sequence to obtain a plurality of sequence reads from corresponding genomic backbone regions and genomic mutation regions of at least one reference genomic sample;
(c) a code sequence to assemble the plurality of sequence reads from the enriched genomic sample and the at least one reference genomic sample; and
(d) a code sequence to determine, based on computational analysis of the assembly, whether the genomic locus has a copy number variation (CNV) or a sequence variation, or a CNV and a sequence variation.
